(19) United States
(12) Patent Application Publication
(10) Pub. No.: US 2022/0219657 A1
(43) Pub. Date: Jul. 14, 2022

(54) SURGICAL TREATMENT INSTRUMENT

(71) Applicant: VOLVO TRUCK CORPORATION, Göteborg (SE)

(72) Inventors: Philippe Alain Lucien Fernand LEGAY, Yville-sur-Seine (FR); David PETROVER, Paris (FR); Frédéric PEYRE, Lasserre (FR)

(73) Assignee: INNOPROD MEDICAL, Plaisance-du-Touch (FR)

(21) Appl. No.: 17/614,144

(22) PCT Filed: May 20, 2020
(86) PCT No.: PCT/EP2020/050850

§ 371 Date: Nov. 24, 2021

(30) Foreign Application Priority Data
May 24, 2019 (FR) 1905492

(51) Int. Cl. B60T 8/1761; B60T 8/1763; B60T 8/175; B60T 8/17

(52) U.S. Cl. CPC B60T 8/17616; B60T 8/17636; B60T 2210/12; B60T 8/1708; B60T 2201/06; B60T 8/175

(57) ABSTRACT

This instrument for surgical treatment, in particular for tenolysis, tenotomy or neurolysis, comprises a main body and a distal part. The distal part has a curved portion forming a hook and is provided with a cutting blade on the inner side of the hook. The main body has a longitudinal lumen for passage of fluid from a proximal end to a distal end of the main body, as well as an elbow with concavity facing the same side as the hook such that the main body has, in the vicinity of its distal end, an injection channel which is in fluidic connection with the lumen via an over-pressure chamber.

The invention relates to an instrument for surgical treatment, in particular for interventions involving the section of fibrous tissue such as tenolysis, tenotomy or neurolysis. The invention also relates to a set comprising a surgical treatment instrument associated with an insertion cannula and/or a syringe. Furthermore, the invention relates to an insertion cannula for a surgical treatment instrument, and to a method of surgical treatment, including cutting fibrous tissue, such as tenolysis, tenotomy or neurolysis, using a surgical treatment instrument.

Tenolysis is a well-known surgical procedure involving the severing of an adhesion formed at a tendon; tenotomy is a surgical procedure involving the severing of a tendon; neurolysis is a surgical procedure involving the severing of an adhesion compressing a nerve. An example of a condition where surgical treatment involves tenolysis is a trigger finger. Specifically, the surgical treatment of the trigger finger includes severing the pulley, i.e. the sheath, that surrounds the flexor tendon, so as to allow release of the flexor tendon from the pulley. An example of a condition where surgical treatment involves neurolysis is carpal tunnel syndrome. Specifically, the surgical treatment of carpal tunnel syndrome involves transection of the anterior annular carpal ligament to allow release of the median nerve from the structures that compress it. Procedures involving the section of fibrous tissue such as tenolysis, tenotomy or neurolysis can be performed for the treatment of different parts of the body, including the hand, shoulder, knee, ankle, foot.

During tenolysis, tenotomy or neurolysis, it is conventional to cut the fibrous tissue with a retro-knife type instrument displaying a sharp hook. The hook of the instrument is first introduced at the front of the fibrous tissue and then the instrument is translated from the front to the back of the fibrous tissue in the direction of an entry incision, so as to sever the fibrous tissue.

Currently, tenolysis, tenotomy and neurolysis are performed:
either by open surgery, with a large incision involving a relatively long and painful postoperative period, requiring a significant amount of resting time;
or by endoscopy, by making a smaller incision and using different instruments for the introduction stage, which is performed blindly, and then for the stage of sectioning the fibrous tissue, which is performed with a vision limited to the endoscope, with relatively long and painful post-operation periods, still requiring a significant amount of resting time.

It is, in particular, to these inconvenience that the invention intends to remedy by proposing an instrument and a method of surgical treatment making it possible to carry out an operation such as tenolysis, tenotomy or neurolysis with a single instrument, in a single gesture and with a single hand, thus being compatible with monitoring by means of the aid of a device held in the other hand, such as an ultrasound probe, while limiting post-operative pain and the duration of downtime.

To this end, according to one aspect, the object of the invention is an instrument for surgical treatment, in particular for tenolysis, tenotomy or neurolysis, comprising a main body and a distal part, wherein the distal part comprises a curved portion forming a hook and is provided with a cutting blade on the inner side of the hook, the main body comprising a longitudinal lumen for the passage of fluid from a proximal end to a distal end of the main body, the main body further comprising an elbow with a concavity turned to the same side as the hook

Preferably, the surgical treatment instrument is configured in such a way that the main body has, in the vicinity of its distal end, an injection channel which is in fluidic connection with the lumen via an over-pressure chamber.

The longitudinal lumen of the main body allows, during a surgical procedure involving the sectioning of fibrous tissue, an overpressure of fluid to be injected in front of the hook so as to spread the soft tissue and produce hydro-dissection when the distal part of the instrument is advanced under the fibrous tissue. In addition, the elbow of the main body provides support for controlled rotation of the instrument, about the elbow forming an axis of rotation, so as to raise the hook towards the fibrous tissue for hooking, prior to severing it by a withdrawal movement of the instrument. Thus, a surgical treatment instrument according to the invention, of which the main body has both a longitudinal fluid passage lumen and a concave elbow facing the same side as the hook, makes it possible to carry out with the same instrument, held in one hand, and in a single movement, both the step of advancing the hook under the fibrous tissue until it protrudes, thanks to the hydro-dissection, and the step of hooking and cutting the fibrous tissue, thanks to the rotational guidance provided by the elbow of the main body, from the position of the hook protruding beyond the fibrous tissue, which guarantees a controlled engagement of the hook with the fibrous tissue before initiating the withdrawal movement generating the cutting of the fibrous tissue.

In one feature, the or each injection channel is configured to direct a stream of fluid radially outwards from the hook of the distal portion.

According to an improvement, the main body has, in the vicinity of its distal end, at least one injection channel in fluidic connection with the lumen of the main body, the or each injection channel being configured to direct a fluid stream radially outwardly from the hook of the distal portion. This arrangement ensures that the over-pressurised fluid streams emitted from the or each injection channel precede the hook, so as to perform hydro-dissection during the hook advancement stage. Preferably, in order not to impede the advancement of the hook under the fibrous tissue, the or each injection channel is configured to direct a stream of fluid radially outwardly from the hook without projecting radially outwardly from the distal portion.

In a preferred embodiment, the over-pressure chamber has a cross-sectional area greater than the cross-sectional area of the main body lumen and the cross-sectional area of each injection channel.

According to one feature, the main body comprises, between the main body lumen and the or each injection channel, said over-pressure chamber of larger cross-section than the cross-section of the main body lumen and the cross-section of each injection channel. Due to the larger cross-sectional area of the over-pressure chamber compared to the cross-sectional area of the or each injection channel and the main body lumen, the over-pressure chamber always maintains a high fluid volume, so that it is instantaneously effective in supplying pressurised fluid to each injection channel and performing hydro-dissection. In particular, the over-pressure chamber allows pressurised fluid to be projected at the or each injection channel with no lag time between an operator's action on a fluid delivery device, such as a syringe, connected with the lumen of the main body at the proximal end of the instrument and the ejection of fluid at the or each injection channel. In addition, the larger volume of the over-pressure chamber limits fluid leakage at the or each injection channel.

According to one feature, the or each injection channel is defined as an extension of an upper portion of the main body lumen, and an upper portion of the over-pressure chamber where present, so as to direct a fluid stream above the hook. An “upper portion” of the main body lumen or over-pressure chamber is defined here as a portion that faces the free end of the hook, whereas a “lower portion” of the main body lumen or over-pressure chamber is a portion that faces the base of the hook. According to an advantageous arrangement, the main body lumen, and the over-pressure chamber when present, are disposed opposite the hook opening extending radially from the base of the hook to the free end of the hook, while the or each injection channel is disposed opposite the hook opening extending radially from a middle portion of the hook to the free end of the hook. Redirection of fluid streams over the hook can thus be achieved without the or each injection channel projecting radially outwardly from the distal part, thereby not disrupting the progress of the hook as it advances under the fibrous tissue.

According to one feature, for the or each injection channel, the ratio of the cross-sectional area of the injection channel to the cross-sectional area of the over-pressure chamber is less than or equal to 0.5, preferably between 0.45 and 0.5. Such a ratio of cross-sectional areas between the or each injection channel and the over-pressure chamber allows an overpressure to be created at each injection channel, such that the streams of fluid propelled from each injection channel are sufficiently powerful to push aside the soft parts and allow hydro-dissection during the hook advancement step.

According to one feature, the ratio of the cross-sectional area of the main body lumen to the cross-sectional area of the over-pressure chamber is less than or equal to 0.7, preferably between 0.6 and 0.7. Such a ratio of cross-sections between the lumen of the main body and the over-pressure chamber allows a high volume of fluid to be maintained in the over-pressure chamber, which has the dual advantage of limiting fluid leakage at the or each injection channel and ensuring optimum responsiveness of the instrument to deliver pressurised fluid at the or each injection channel, without latency.

According to one feature, the elbow defines on the concave side an angle of between 90° and 160°, preferably of the order of 120°, between a portion of the main body upstream of the elbow and a portion of the main body downstream of the elbow. Such an angle between the portions upstream and downstream of the elbow is optimal to allow the practitioner to easily and reliably rotate the instrument around the elbow from the position of the hook protruding beyond the fibrous tissue, so that the hook hook hooks the fibrous tissue directly through this rotation.

According to one feature, the cutting blade of the hook comprises an inner blade portion, located inside the hook, and an upstream blade portion, located upstream of the hook, the upstream blade portion being inclined towards the inside of the hook, in particular at an angle of between 5° and 30°, relative to the longitudinal axis of the distal portion. This inclination of the upstream blade portion makes it possible, once the fibrous tissue has been hooked using the hook, to have the upstream blade portion oriented transversely, i.e. at an angle, with respect to the tissue and in engagement with it. This makes it possible to cut the fibrous tissue not only by means of the inner blade portion, but also by means of the upstream blade portion during the withdrawal movement of the instrument, which is not the case with an upstream blade portion parallel to the longitudinal axis of the distal portion or inclined towards the outside of the hook. This results in improved cutting efficiency of the instrument and reduced effort required by the practitioner to cut the fibrous tissue during the withdrawal movement of the instrument.

The inclination of the upstream blade portion is particularly effective in combination with the main body elbow. Indeed, thanks to the elbow of the main body, the instrument according to the invention makes it possible to impose a fixed inclination of the distal part with respect to the fibrous tissue during the withdrawal movement, which makes it possible to guarantee an optimal grip of the fibrous tissue between the inner blade part and the upstream blade part.

The cumulative length of the main body portion downstream of the elbow and distal part is usually fixed for a given application. For example, in the case of wrist or finger procedures, such as the treatment of carpal tunnel syndrome or trigger finger, the cumulative length of the main body portion downstream of the elbow and the distal part is typically between 15 mm and 80 mm.

According to one feature, the length of the portion of the main body upstream of the elbow can be modulated so as to adjust the lever arm, and thus the force to be applied by the practitioner with the instrument, to sever the fibrous tissue during the withdrawal movement of the instrument. In particular, in the case of wrist or finger procedures, especially in the treatment of carpal tunnel syndrome or jerk finger, the length of the main body portion upstream of the elbow is typically between 10 mm and 100 mm.

In one feature, the surgical treatment instrument comprises a tip configured to co-operate with a syringe, so as to bring the syringe body into fluidic connection with the lumen of the main body at the proximal end of the main body. It is thus possible to inject a fluid under overpressure from the syringe body towards the distal part of the instrument, by acting on the plunger of the syringe, to perform the hydro-dissection at the front of the hook during the step of advancing the hook under the fibrous tissue. Advantageously, the tip of the surgical treatment instrument is a male or female tip, in particular of the Luer Lock type, configured to cooperate with a complementary female or male tip, in particular of the Luer Lock type, of the syringe.

In one feature, the rotation of the surgical treatment instrument around the elbow is actuated by the practitioner by acting on a syringe which is linked to the proximal end of the instrument and forms an actuating handle. Advantageously, the practitioner can then not change his grip on the syringe body between the step of advancing the hook under the fibrous tissue and the step of hooking and severing the fibrous tissue: in particular, in the step of advancing the hook under the fibrous tissue, the practitioner can hold the syringe body with one hand and act with the same hand on the plunger of the syringe to perform the hydro-dissection; then, in the step of hooking and severing the fibrous tissue, the practitioner may maintain his or her grip on the syringe body with the same hand and rotate the instrument around the elbow to bring the hook into engagement with the fibrous tissue at the front of the fibrous tissue before initiating the withdrawal movement of the instrument still acting on the syringe body. Alternatively, of course, the practitioner may choose to change the grip between the step of advancing the hook under the fibrous tissue and the step of hooking and severing the fibrous tissue, including holding the syringe body to perform the hydro-dissection during the step of advancing the hook, and then preferring to hold the main body of the instrument to rotate the instrument around the elbow and actuate the withdrawal movement of the instrument during the step of hooking and severing the fibrous tissue.

In one embodiment of the invention, the distal portion of the surgical treatment instrument has a sharp distal end, allowing an entry incision to be made for the introduction of the instrument. An instrument having such a sharp distal end is used when the interventional site does not involve the risk of severing nerves, vessels or other vulnerable structures which it is important not to sever, which is the case for example for the surgical treatment of the trigger finger.

In another embodiment of the invention, the distal portion of the surgical treatment instrument has a rounded distal end. An instrument with such a rounded distal end is used when the interventional site involves risks of severing nerves, vessels or other vulnerable structures that are important not to be severed, which is the case for example for the surgical treatment of carpal tunnel syndrome where the flexor tendons of the fingers and the median nerve running through the carpal tunnel must be preserved. The instrument is then advantageously associated with an insertion cannula having a sharp distal end, allowing an entry incision to be made for the introduction of the instrument under the skin, the rounded distal end of the instrument then being moved from the insertion cannula to advance under the fibrous tissue to be severed without the risk of severing surrounding vulnerable structures.

In one feature, the main body and the distal portion of the surgical treatment instrument are configured to cooperate with an insertion cannula having a sharp, pointed distal end, the insertion cannula comprising a tubular body defining a housing for receiving the distal portion and an elbow rest plate of the main body.

According to one feature, the support plate of the insertion cannula has a length, taken in the longitudinal direction of the tubular body, equal to or greater than the length of a fibrous tissue to be cut with the surgical treatment instrument. Thus, the support plate provides a support of sufficient length so that the portion of the main body downstream of the elbow can slide systematically on this support plate, without direct contact on the skin, during the step of advancing or withdrawing the hook in the instrument insertion position. Furthermore, this length of the support plate prevents any injury to the skin during the instrument withdrawal movement, which could result from the support plate moving backwards if it were too short. For example, in an embodiment especially adapted for the surgical treatment of carpal tunnel syndrome, the length of the support plate of the insertion cannula, taken in the longitudinal direction of the tubular body, is greater than or equal to the length of the tendon area of the carpal tunnel. Preferably, a safety margin is provided for the length of the support plate, i.e. the length of the support plate is greater, in particular in the range of 10% to 50%, than the length of the fibrous tissue to be severed with the surgical treatment instrument.

According to one feature, the support plate of the insertion cannula comprises:
a median part for supporting the elbow of the surgical treatment instrument, which is arranged in the extension of the tubular body, and
two lateral wings on either side of the middle part, which are capable of being bent towards each other on the side of the tubular body.

The median part of the support plate forms a support allowing not only the portion of the main body downstream of the elbow to slide without direct contact on the skin during the step of advancing or retracting the hook in the insertion position of the instrument, but also the elbow to be supported during the pivoting of the instrument. The lateral wings of the support plate are intended to be raised so as to surround the main body of the instrument and allow the main body to be gripped to initiate the withdrawal movement of the instrument generating the section of fibrous tissue.

According to one aspect, which may be considered independently of the features described above, the invention has as its object an instrument for surgical treatment, in particular for tenolysis, tenotomy or neurolysis, comprising a main body and a distal part, wherein the distal part has a curved portion forming a hook and is provided with a cutting blade on the inner side of the hook, the main body having a longitudinal lumen for the passage of fluid from a proximal end to a distal end of the main body, the main body of the instrument also comprising, in the vicinity of its distal end, at least one injection channel in fluidic connection with the lumen of the main body, the or each injection channel being configured to direct a stream of fluid radially outwards with respect to the hook of the distal part, preferably without projecting radially outwards with respect to the distal part. In particular, according to one embodiment, the or each injection channel is defined as an extension of an upper portion of the lumen of the main body, and of a possible overpressure chamber, so as to direct a fluid stream above the hook.

The invention also relates to an assembly comprising a surgical treatment instrument as described above and an insertion cannula having a sharp distal end, the insertion cannula comprising a tubular body defining a housing for receiving the distal part of the instrument in the insertion position, and a support plate for the main body elbow in the cutting position.

The invention also relates to an assembly comprising a surgical treatment instrument as described above and a syringe whose syringe body is in fluidic connection with the lumen of the main body at the proximal end.

The invention also relates to an assembly comprising a surgical treatment instrument and an insertion cannula in which the support plate has a length, in the longitudinal direction of the tubular body, greater than or equal to the length of a fibrous tissue to be severed.

According to another embodiment, the invention also relates to an assembly comprising a surgical treatment instrument in which the support plate comprises a median elbow support portion in the extension of the tubular body and two lateral wings on either side of the median portion which are capable of being folded towards each other on the side of the tubular body.

According to one aspect, the invention relates to an insertion cannula intended to cooperate with a surgical treatment instrument comprising a main body provided with an elbow and a distal part provided with a cutting hook, the concavity of the elbow being turned to the same side as the cutting hook, the insertion cannula having a pointed and cutting distal end, the insertion cannula comprising a tubular body defining a housing for receiving the distal portion of the surgical treatment instrument and a support plate of the elbow of the main body of the surgical treatment instrument, wherein the support plate has a length, taken in the longitudinal direction of the tubular body, equal to or greater than the length of a fibrous tissue to be severed with the surgical treatment instrument. The support plate thus provides a support of sufficient length so that the portion of the main body downstream of the elbow of the instrument can slide systematically over this support plate, without direct contact with the skin, during the step of advancing or withdrawing the hook in the instrument insertion position. In addition, this length of the support plate prevents any injury to the skin during the instrument withdrawal movement, which could result from the support plate moving backwards if it were too short.

According to a feature of the insertion cannula, the support plate comprises a middle part for supporting the elbow in the extension of the tubular body, and two lateral wings on either side of the middle part which are capable of being bent towards each other on the side of the tubular body.

The invention also relates to a method of surgical treatment, including severing fibrous tissue, such as tenolysis, tenotomy or neurolysis, using a surgical treatment instrument as described above, the method comprising steps in which:
a syringe is assembled with the main body of the instrument, such that the syringe is in fluidic connection with the lumen of the main body at the proximal end of the main body;
in the instrument insertion position, in which the portion of the main body downstream of the elbow is parallel to the fibrous tissue to be severed, the distal part of the instrument is inserted through an entry incision and the distal part of the instrument is engaged under the fibrous tissue to be severed until the hook protrudes from it, the engagement of the distal part of the instrument under the fibrous tissue being achieved by hydro-dissection using a fluid contained in the syringe and injected in front of the distal end of the instrument, by activating the piston of the syringe;
from the position of the hook projecting beyond the fibrous tissue to be cut, the instrument is pivoted about the elbow to a cutting position, in which the portion of the main body downstream of the elbow is transverse to the fibrous tissue to be cut, so that the hook is raised opposite the fibrous tissue to be cut;
the instrument is retracted so that the fibrous tissue to be cut is hooked by the hook and cut by the blade of the distal part;
once the fibrous tissue has been severed, the instrument is repivoted around the elbow in the opposite direction to the insertion position and the instrument is extracted through the entry incision.

According to an embodiment, when the distal portion of the surgical treatment instrument is inserted using an insertion cannula, the method comprises steps in which:
a syringe is assembled with the main body of the instrument, such that the syringe is in fluidic connection with the lumen of the main body at the proximal end;
an entry incision is made with the sharp distal end of the insertion cannula;
the distal part of the instrument is inserted into the housing of the tubular body of the insertion cannula so that the hook is received in the housing;
the distal part of the instrument is inserted through the entry incision using the insertion cannula with the instrument in the insertion position;
the distal part of the instrument is engaged under the fibrous tissue to be cut by sliding the portion of the main body downstream of the elbow on the support plate of the insertion cannula until the hook protrudes beyond the fibrous tissue to be cut, the engagement of the distal part of the instrument under the fibrous tissue being carried out by hydro-dissection with the aid of a fluid contained in the syringe and injected in front of the distal end of the instrument, by actuating the plunger of the syringe;
from the position of the hook projecting beyond the fibrous tissue to be cut, the instrument is pivoted around the elbow to the cutting position, supported on the support plate, so that the hook is raised opposite the fibrous tissue to be cut;
the instrument is withdrawn by holding the main body with the support plate of the insertion cannula, so that the fibrous tissue to be cut is hooked by the hook and cut by the blade of the distal part;
once the fibrous tissue has been cut, the instrument is rotated around the elbow in the opposite direction towards the insertion position, supported by the support plate;
the portion of the main body downstream of the elbow is slid over the support plate of the insertion cannula until the hook is received in the housing of the tubular body of the insertion cannula;
the instrument and insertion cannula are extracted through the entry incision while the hook is received in the tubular body of the insertion cannula.

The features and advantages of the present invention will become apparent from the following description of two embodiments of an instrument and method of surgical treatment according to the invention, given by way of example only and made with reference to the attached drawings, in which:

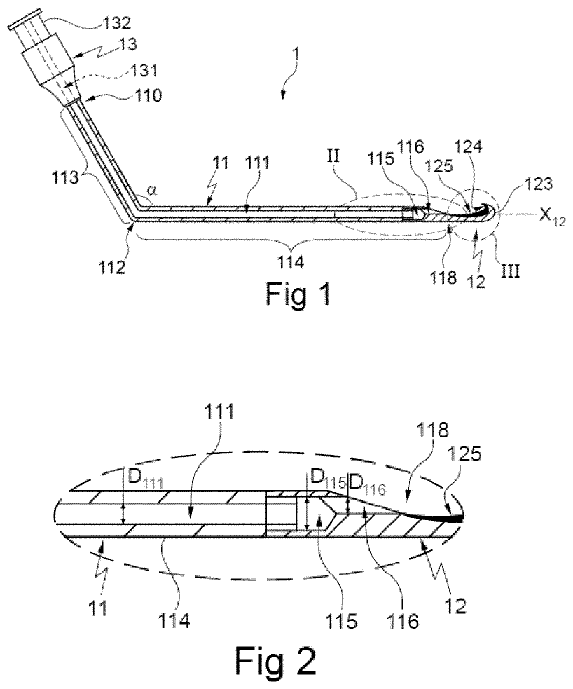

FIG. 1 is a cross-section of a surgical treatment instrument according to a first embodiment of the invention;

FIG. 2 is a larger scale view of detail II of FIG. 1;

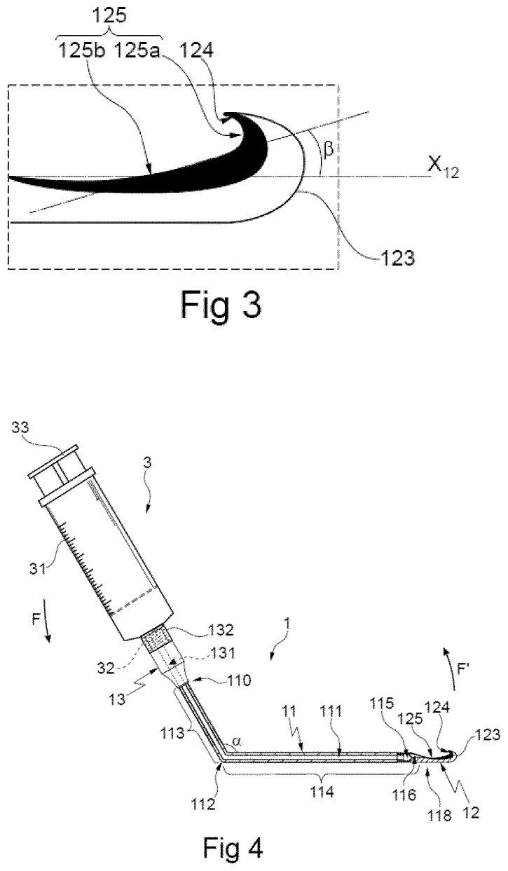

FIG. 3 is a larger scale view of detail III of FIG. 1;

FIG. 4 is a cross-section analogous to FIG. 1, with the surgical treatment instrument associated with a syringe;

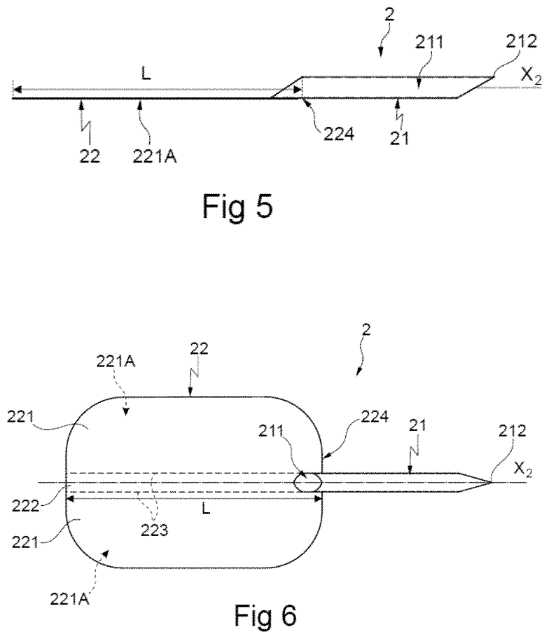

FIG. 5 is a cross-section of an insertion cannula adapted to cooperate with the surgical treatment instrument of FIGS. 1 to 4;

FIG. 6 is a top view of the insertion cannula of FIG. 5;

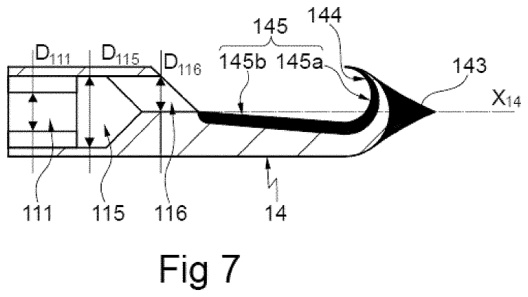

FIG. 7 is a cross-section of the distal portion of a surgical treatment instrument according to a second embodiment of the invention.

FIG. 1 shows a surgical treatment instrument 1, of the type retroknife with a rounded distal end. By way of example, the instrument 1 of this first embodiment is intended for the surgical treatment of carpal tunnel syndrome. The instrument 1 comprises a main body 11, a distal portion 12, and a proximal tip 13 configured to cooperate with a syringe. Advantageously, the tip 13 is a male luer lock fitting comprising a male connector 132 configured to cooperate with a connector 32 complementary to a female luer lock fitting fitted to a syringe 3. The instrument 1 is thus compatible with conventional syringes equipped with female luer lock fittings, which are readily available.

The distal part 12 of the instrument has a curved portion forming a hook 124, and is provided with a cutting blade 125 on the inner side of the hook 124. In addition, the main body 11 has a concave elbow 112 facing the same side as the hook 124, which defines on the concave side an angle of about 120° between an upstream portion 113 of the main body located upstream of the elbow 112 and a downstream portion 114 of the main body located downstream of the elbow 112.

The main body 11 of the instrument also has a longitudinal lumen 111 for the passage of fluid from a proximal end 110 to a distal end 118 of the main body 11, which is continued into the tip 13 by a corresponding lumen 131. As is clearly visible in FIG. 2, the main body 11 has, in the vicinity of its distal end 118, an injection channel 116 which is in fluidic connection with the lumen 111 via an over-pressure chamber 115.

In this embodiment, each one of the lumen 111, the over-pressure chamber 115 and the injection channel 116 is circular in cross-section, being understood that different cross-sections may be envisaged within the scope of the invention. The over-pressure chamber 115 has a diameter Di 15 greater than the diameter D₁₁₁ of the lumen 111 of the main body and the diameter Di 16 of the injection channel 116. Thus, having a larger cross-sectional area than those of the main body lumen 111 and the injection channel 116, the over-pressure chamber 115 is configured to maintain a high volume of fluid at all times, so that it is instantaneously effective in delivering pressurised fluid to the injection channel 116, without latency. In addition, the higher volume of the over-pressure chamber 115 helps to limit uncontrolled fluid leakage at the injection channel 116.

The ratio of the diameter D₁₁₆ of the injection channel 116 to the diameter D₁₁₅ of the over-pressure chamber 115 is in the range of 0.45 to 0.5. This creates an overpressure at the injection channel 116 level, so as to generate powerful streams of fluid from the injection channel. The ratio of the diameter D₁₁₁ of the main body lumen 111 to the diameter D₁₁₅ of the over-pressure chamber is in the range of 0.6 to 0.7. This allows a high volume of fluid to be maintained in the over-pressure chamber 115, with the dual advantage of limiting uncontrolled fluid leakage at the injection channel 116 and ensuring optimal responsiveness of the instrument 1 to deliver pressurised fluid at the injection channel 116, without latency. In particular, by way of non-limiting example, the over-pressure chamber 115 has a diameter D₁₁₅ of 2.2 mm; the lumen 111 of the main body 11 has a diameter D₁₁₁ of 1.4 mm; the injection channel 116 has a diameter D₁₁₆ of 1.06 mm.

As is clearly visible in FIG. 2, the injection channel 116 is defined as an extension of an upper portion of the lumen 111 and an upper portion of the over-pressure chamber 115, so as to direct a fluid stream above the hook 124. In this way, redirection of fluid streams above the hook 124 is achieved without the channel 116 projecting radially outwardly from the distal portion 12, thereby not disrupting the progress of the hook 124 as it advances under the fibrous tissue. The longitudinal lumen 111 of the main body makes it possible, during a surgical procedure involving the sectioning of fibrous tissue, to inject a fluid under overpressure in front of and above the hook 124 so as to spread the soft parts and produce a hydro-dissection during the progression of the distal part 12 of the instrument under the fibrous tissue.

Furthermore, the elbow 112 of the main body forms an axis of rotation allowing controlled rotation of the instrument 1, so as to raise the hook 124 towards the fibrous tissue in order to hook it, before cutting it by a withdrawal movement of the instrument 1. Thus, the instrument 1 according to the invention, whose main body has both a longitudinal lumen 111 for the passage of fluid and an elbow 112 with a concavity turned to the same side as the hook 124, makes it possible to carry out with the same instrument, held in one hand, and in a single movement, both the step of advancing the hook 124 under the fibrous tissue thanks to hydro-dissection, and the step of hooking and sectioning the fibrous tissue thanks to the rotational guidance provided by the elbow 112. The cutting blade 125 comprises an inner blade portion 125a, located internally in the bottom of the hook 124, and an upstream blade portion 125b, located upstream of the hook 124. As is clearly visible in FIG. 3, the upstream blade portion 125b is inclined towards the interior of the hook at an angle β of the order of 25° with respect to the longitudinal axis X₁₂ of the distal portion 12. This inclination of the upstream blade portion 125b allows, once the fibrous tissue is hooked with the hook 124, the upstream blade portion 125b to be oriented transversely with respect to the fibrous tissue so as to be able to attack the cutting of the fibrous tissue at an angle rather than horizontally. The cutting of the fibrous tissue is thus carried out by means of both the inner blade portion 125a and the upstream blade portion 125b during the withdrawal movement of the instrument 1. The inclination of the upstream blade portion 125b is particularly effective in combination with the elbow 112 of the main body 11. This is because the elbow 112 allows a fixed inclination of the distal portion 12 to be imposed with respect to the fibrous tissue during the withdrawal movement, thereby ensuring an optimal grip of the fibrous tissue between the inner blade portion 125a and the upstream blade portion 125b.

For example, in this first embodiment intended for the surgical treatment of carpal tunnel syndrome, the cumulative length of the downstream portion 114 of the main body and of the distal part 12 is around 75 mm, which corresponds to the morphology of the carpal tunnel. The length of the upstream portion 113 of the main body 11 can be modulated to adjust the lever arm desired by the surgeon, and therefore the force to be applied to cut the fibrous tissue during the withdrawal movement of the instrument 1. In the context of the treatment of carpal tunnel syndrome, the length of the upstream portion 113 of the main body 11 is typically in the range of 10 mm to 100 mm.

Alternatively, the practitioner may choose not to change his or her grip on the body 31 of the syringe 3 between the step of advancing the hook 124 under the fibrous tissue and the step of hooking and severing the fibrous tissue; or the practitioner may choose to change his or her grip between the step of advancing the hook 124 under the fibrous tissue and the step of hooking and severing the fibrous tissue, in particular he or she may hold the body 31 of the syringe to perform the hydro-dissection during the step of advancing the hook 124, and then prefer to hold the upstream portion 113 of the main body 11 of the instrument in order to rotate the instrument around the elbow 112 and actuate the retraction motion of the instrument during the step of hooking and severing the fibrous tissue. The selection of either option may depend, in particular, on the length of the upstream portion 113 of the main body 11.

In this first embodiment, the distal portion 12 of the instrument 1 has a rounded distal end 123. An instrument 1 with such a rounded distal end is used in particular when nerves, vessels or other vulnerable structures that it is important not to hurt are present in the vicinity of the fibrous tissue to be cut, as is the case, for example, of an operation on the carpal tunnel. For its insertion under the skin, the instrument 1 is then advantageously associated with an insertion cannula 2 as shown in FIGS. 5 and 6, having a sharp distal end 212 allowing to make an entry incision for the introduction of the instrument 1. Once inserted under the skin, the rounded distal end 123 of the instrument 1 can be moved from the insertion cannula 2 to advance under the fibrous tissue to be severed without the risk of severing surrounding vulnerable structures. In this first embodiment, by way of example, the instrument 1 and the insertion cannula 2 are made of a biocompatible metal, in particular stainless steel.

The insertion cannula 2 comprises a tubular body 21 of axis X₂, which delimits a housing 211 for receiving the distal part 12 of the instrument 1, and a support plate 22, which is intended to receive the elbow 112 and the downstream part 114 of the main body 11 of the instrument 1 in a supporting manner. The distal edge 224 of the support plate 22 forms an insertion limit of the insertion cannula 2 under the skin.

Furthermore, the support plate 22 has a length L, taken in the longitudinal direction of the tubular body 21, greater than or equal to the length of the fibrous tissue to be cut with the instrument 1. Thus, it is ensured that the support plate 22 provides a support of sufficient length so that the downstream portion 114 of the main body 11 can slide systematically without direct contact on the skin during the step of advancing and retracting the hook 124 in the position of insertion of the instrument 1. Moreover, such a length of the support plate 22 makes it possible to avoid injuries to the skin during the movement of withdrawal of the instrument 1, which could result from a recoil of the support plate if it were too short. For example, for an insertion cannula 2 adapted for the surgical treatment of carpal tunnel syndrome, the length L of the support plate 22 is equal to the length of the tendon zone of the carpal tunnel plus a safety margin of 50%, i.e. about 45 mm.

As can be seen in FIG. 6, the support plate 22 of the insertion cannula 2 comprises a median part 222, which is arranged in the extension of the tubular body 21, and two lateral wings 221 on either side of the median part 222, which are capable of being folded towards each other on the side of the tubular body 21 according to predefined folding lines 223. The median part 222 forms a support allowing the downstream portion 114 of the main body 11 to slide without direct contact on the skin during the step of advancing and withdrawing the hook 124 in the insertion position of the instrument 1, and the elbow 112 to be supported during the pivoting of the instrument 1. The lateral wings 121 of the support plate, when they are raised so as to surround the main body 11 of the instrument 1, allow the main body 11 to be gripped, in particular between the thumb and forefinger of the practitioner, on the side of their face 221A opposite the tubular body 21 (lower face in FIG. 6), in order to initiate the withdrawal movement of the instrument 1 generating the sectioning of the fibrous tissue.

An example of a method of surgical treatment using the instrument 1 in combination with the insertion cannula 2 comprises steps as described below. As a non-limiting example, the method described below can be implemented for the treatment of carpal tunnel

First of all, a syringe 3, of which the syringe body 31 has been previously filled with a hydro-dissecting liquid such as a sterile saline solution, is assembled with the main body 11 of the instrument 1, in such a way that the syringe body 31 is in fluidic connection with the lumen 111 of the main body 11 at the proximal end 110. With reference to FIG. 4, this is achieved by connecting the male Luer Lock connector 132 of the tip 13 of the instrument 1 with the complementary female Luer Lock connector 32 of the syringe 3.

An entry incision is then made with the distal end 212 of the insertion cannula 2 and then, after purging the instrument 1 with the filled syringe thereon, the distal part 12 of the instrument 1 is inserted into the housing 211 of the tubular body 21 of the insertion cannula 2, so that the hook 124 is received in the housing 211. In doing so, the distal part 12 of the instrument 1 is inserted through the entry incision, the instrument 1 being in an insertion position in which the downstream portion 114 of the main body 11 is parallel to the fibrous tissue to be severed.

The downstream portion 114 of the main body 11 is then slid over the medial part 222 of the insertion cannula support plate 22, so as to engage the distal part 12 of the instrument 1 under the fibrous tissue to be severed. The engagement of the distal part 12 is achieved by spreading the tissue by hydro-dissection, using the hydro-dissecting fluid injected in front of the distal end 123 of the instrument 1 from the injection channel 116, thanks to the actuation of the piston 33 of the syringe 3. The advancement of the distal portion 12 continues until the hook 124 protrudes beyond the fibrous tissue to be cut. Advantageously, the advancement of the hook 124 under the fibrous tissue to be cut is controlled by means of an ultrasound probe held in one hand, while the other hand actuates both the advancing movement of the instrument 1 and the advancing movement of the plunger 33 while holding the syringe 3.

From the position of the hook 124 projecting beyond the fibrous tissue to be cut, the instrument 1 is pivoted in the direction of arrow in FIG. 4, about the elbow 112 resting on the median part 222 of the support plate 22, towards a cutting position in which the downstream portion 114 of the main body 11 is transverse to the fibrous tissue to be cut. Thus, the hook 124 is raised opposite the fibrous tissue to be cut. Advantageously, this pivoting of the instrument 1 towards the cutting position can be actuated by acting on the syringe 3, which acts as a handle, in the direction of the arrow F in FIG. 4. Alternatively, the practitioner may prefer to hold the upstream portion 113 of the main body 11 to pivot the instrument 1 about the elbow 112.

A withdrawal movement of the instrument 1 is then carried out, holding the main body 11 with the help of the side wings 221 of the support plate 22 of the insertion cannula 2, folded towards each other on the side of the tubular body 21, so that the fibrous tissue to be cut is hooked by the hook 124 and cut by the blade 125. More precisely, during the withdrawal movement of the instrument 1, the fibrous tissue is engaged between the inner blade portion 125a and the upstream blade portion 125b, both of which participate in cutting the fibrous tissue. Once the fibrous tissue has been cut, the instrument 1 is pivoted back around the elbow 112 in the opposite direction, i.e. in the direction opposite to the arrow F in FIG. 4, towards the insertion position by resting on the middle part 222 of the support plate 22.

Finally, the downstream portion 114 of the main body 11 is slid over the middle part 222 of the support plate 22 of the insertion cannula 2, until the hook 124 is received in the housing 211 of the tubular body 21 of the insertion cannula 2, and the instrument 1 and the insertion cannula 2 are extracted through the entry incision while the hook 124 is received in the housing 211.

In the second embodiment shown in FIG. 7, the elements similar to those of the first embodiment have identical references. By way of example, the instrument 1 of this second embodiment is intended for the surgical treatment of the protruding finger. The instrument 1 of this second embodiment differs from that of the first embodiment by its distal portion 14, which has a sharp, pointed distal end 143 and a differently profiled cutting blade 145, as well as by the respective diameters of the lumen 111, the over-pressure chamber 115 and the injection channel 116.

Similar to the first embodiment, the distal part 14 has a curved portion forming a hook 144, and is provided with a cutting blade 145 on the inner side of the hook 144. The sharp, pointed distal end 143 has the advantage that an entry incision for the introduction of the instrument 1 can be made directly with the instrument 1 itself, without the need for a complementary insertion cannula. However, an instrument 1 with such a sharp distal end is only used when the interventional site does not involve the risk of severing nerves, vessels or other vulnerable structures, which is the case, for example, for the surgical treatment of the jerk finger. The instrument 1 of the second embodiment also has the same advantages as the instrument 1 of the first embodiment.

In this second embodiment, by way of example, the instrument 1 is made of a biocompatible metal, in particular stainless steel. As in the first embodiment, the ratio of the diameter D₁₁₆ of the injection channel 116 to the diameter D₁₁₅ of the over-pressure chamber 115 is in the range of 0.45 to 0.5, and the ratio of the diameter D₁₁₁ of the lumen 111 of the main body to the diameter D₁₁₅ of the over-pressure chamber is in the range of 0.6 to 0.7. In particular, by way of non-limiting example, the over-pressure chamber 115 has a diameter D₁₁₅ of 1.6 mm; the lumen 111 of the main body 11 has a diameter D₁₁₁ of 1 mm; the injection channel 116 has a diameter D₁₁₆ of 0.8 mm.

As in the first embodiment, the cutting blade 145 comprises an inner blade portion 145a, located internally in the bottom of the hook 144, and an upstream blade portion 145b, located upstream of the hook 144. It should be noted that, in the second embodiment, the upstream blade portion 145b is slightly inclined outwardly of the hook 144 with respect to the longitudinal axis X14 of the distal portion 14, instead of being inclined inwardly of the hook as in the first embodiment. Of course, alternatively, the upstream blade portion 145b may be inclined towards the inside of the hook, in particular at an angle of between 5° and 30°, relative to the longitudinal axis of the distal portion 14, to allow, once the fibrous web is hooked with the hook, the upstream blade portion 145b to be oriented transversely to the fibrous web so that it can engage the cut of the fibrous web at an angle rather than horizontally.

An example of a method of surgical treatment using the instrument 1 of the second embodiment, without using an insertion cannula, comprises steps as described below. As a non-limiting example, the method described below can be implemented for the treatment of the trigger finger, where the fibrous tissue to be severed is a pulley surrounding a flexor tendon.

First of all, a syringe 3, the syringe body 31 of which has been previously filled with a hydro-dissecting liquid such as a sterile saline solution, is assembled with the main body 11 of the instrument 1, so that the syringe body 31 is in fluidic connection with the lumen 111 of the main body 11 at the proximal end 110.

After purging the instrument 1 with the filled syringe on top, an entry incision is then made with the sharp distal end 143 and, in the insertion position of the instrument 1 in which the downstream portion 114 of the main body 111 is parallel to the fibrous tissue to be cut, the distal part 14 of the instrument is inserted through the entry incision.

The downstream portion 114 of the main body 11 is then advanced to engage the distal part 14 of the instrument 1 under the fibrous tissue to be cut. The engagement of the distal portion 14 is achieved by hydro-dissecting the tissue apart, using hydro-dissecting fluid injected at the front of the distal end 143 of the instrument 1 from the injection channel 116, through the operation of the plunger 33 of the syringe 3. The advancement of the distal portion 14 continues until the hook 144 protrudes beyond the fibrous tissue to be severed. Advantageously, the advancement of the hook 144 under the fibrous tissue to be cut is controlled by means of an ultrasound probe held in one hand, while the other hand actuates both the advancing movement of the instrument 1 and the advancing movement of the plunger 33 while holding the syringe 3.

From the position of the hook 144 projecting beyond the fibrous tissue to be cut, the instrument 1 is rotated about the elbow 112 in a direction similar to that of arrow F in FIG. 4, to a cutting position in which the downstream portion 114 of the main body 11 is transverse to the fibrous tissue to be cut. Thus, the hook 144 is raised opposite the fibrous tissue to be cut.

The instrument 1 is then withdrawn, in particular by holding the syringe body 31 or the main body 11, so that the fibrous tissue to be cut is hooked by the hook 144 and cut by the blade 145. Once the fibrous tissue has been severed, the instrument 1 is pivoted back around the elbow 112 in the opposite direction to that of arrow F in FIG. 4, towards the insertion position, and the instrument 1 is withdrawn through the entry incision.

As can be seen from the preceding examples, whatever the mode of implementation, thanks to the presence of the lumen 111 and the elbow 112 of the main body, a surgical treatment instrument according to the invention makes it possible to carry out with the same instrument, held with one hand, and in a single gesture both the step of advancing the hook 124, 144 under the fibrous tissue thanks to the hydro-dissection, and the step of hooking and sectioning the fibrous tissue thanks to the rotational guidance provided by the elbow 112, without the need to previously incise the skin with a scalpel to introduce the instrument. Very advantageously, a surgical treatment instrument according to the invention is compatible with monitoring using a device, for example an ultrasound probe, held in the other hand than the one operating the instrument. More generally, a surgical treatment instrument according to the invention allows better controlled interventions to be performed, which limits post-operative pain for the patient and the duration of downtime.

The invention is not limited to the examples described and shown.

In particular, in the preceding examples, the invention has been described for the sectioning of fibrous tissues of the wrist or finger, especially in the context of the treatment of carpal tunnel syndrome or trigger finger. However, a surgical treatment instrument and an insertion cannula according to the invention can be used for the sectioning of any fibrous tissue, in particular fibrous tissue of the hand, shoulder, knee, ankle or foot, the dimensions of the instrument and of the insertion cannula then being adapted on a case-by-case basis to correspond to the length and the environment of the fibrous tissue to be sectioned.

Generally speaking, the geometry of a surgical treatment instrument according to the invention may be different from those described above. In particular: the cumulative length of the portion of the main body downstream of the elbow and of the distal part, if substantially fixed for a given application, tends to vary from one application to another; the length of the portion of the main body downstream of the elbow can be modulated so as to adjust the lever arm and thus the force to be applied by the practitioner to sever the fibrous tissue during the withdrawal movement of the instrument; or the respective diameters of the lumen of the main body, the over-pressure chamber and the injection channel(s) can be adjusted to optimise the hydro-dissection. A surgical treatment instrument and an insertion cannula according to the invention may also be made of any materials suitable for their function, in particular of biocompatible metal and/or polymer. The use of a non-magnetic material, in particular a polymer, is advantageous for enabling MRI (Magnetic Resonance Imaging) monitoring of an operation involving a surgical treatment instrument and/or an insertion cannula according to the invention.

1.-15. (canceled)

16. A surgical treatment instrument for tenolysis, tenotomy or neurolysis, comprising:
a main body comprising a longitudinal lumen for passage of fluid from a proximal end to a distal end of the main body, and
a distal part, wherein the distal part has a curved portion forming a hook and is provided with a cutting blade on the inner side of the hook,
the main body further comprising an elbow presenting a concavity facing the same side as the hook,
wherein the main body has, in the vicinity of its distal end:
an over-pressure chamber,
an injection channel,
the injection channel being in fluidic connection with the lumen via the over-pressure chamber, the injection channel being configured to direct a fluid stream above the hook,
the over-pressure chamber being configured to delivering pressurised fluid to the injection channel.

17. The surgical treatment instrument according to claim 16, wherein the or each injection channel is configured to direct a stream of fluid radially outwardly from the hook of the distal portion.

18. The surgical treatment instrument according to claim 17, wherein the over-pressure chamber has a cross-sectional area greater than the cross-sectional area of the lumen of the main body and the cross-sectional area of each injection channel.

19. The surgical treatment instrument according to claim 17, wherein the or each injection channel is defined as an extension of an upper portion of the lumen of the main body, and an upper portion of the over-pressure chamber where present, so as to direct a stream of fluid above the hook.

20. The surgical treatment instrument according to claim 18, wherein the ratio of the cross-sectional area of the lumen of the main body to the cross-sectional area (Dias) of the over-pressure chamber is less than or equal to 0.7, preferably between 0, 6 to 0.7, and wherein for the or each injection channel the ratio of the cross-sectional area of the injection channel to the cross-sectional area of the over-pressure chamber is less than or equal to 0.5, preferably between 0.45 and 0.5.

21. The surgical treatment instrument according to claim 16, wherein the elbow defines on the concave side an angle of between 90° and 160°, preferably of the order of 120°, between a portion of the main body upstream of the elbow and a portion of the main body downstream of the elbow.

22. The surgical treatment instrument according to claim 16, wherein the cutting blade of the hook comprises an inner blade portion inside the hook and an upstream blade portion upstream of the hook, the upstream blade portion being inclined towards the inside of the hook, in particular at an angle of between 5° and 30°, relative to the longitudinal axis of the distal portion.

23. The surgical treatment instrument according to claim 16, comprising a tip configured to cooperate with a syringe so as to bring the syringe body into fluidic connection with the lumen of the main body at the proximal end.

24. The surgical treatment instrument according to claim 16, wherein the distal portion has a sharp distal end.

25. The surgical treatment instrument according to claim 16, wherein the distal portion has a rounded distal end.

26. The surgical treatment instrument according to claim 25, wherein the main body and the distal portion are configured to cooperate with an insertion cannula having a sharp distal end, the insertion cannula comprising a tubular body delimiting a housing for receiving the distal portion and a support plate for the elbow of the main body.

27. An assembly comprising a surgical treatment instrument according claim 16, an insertion cannula having a sharp distal end, the insertion cannula comprising a tubular body delimiting a housing for receiving the distal part, and a plate for supporting the elbow of the main body.

28. An assembly comprising a surgical treatment instrument according to claim 16 and a syringe in fluidic connection with the lumen of the main body at the proximal end.

29. A set comprising a surgical treatment instrument according to claim 26, and an insertion cannula wherein the support plate has a length, in the longitudinal direction of the tubular body equal to or greater than the length of a fibrous tissue to be severed.

30. The set comprising a surgical treatment instrument according to claim 29, wherein the support plate comprises a median part for supporting the elbow in the extension of the tubular body and two lateral wings on either side of the median part, which are capable of being folded towards each other on the side of the tubular body.
